(19) United States
(12) Patent Application Publication
(10) Pub. No.: US 2025/0074947 A1
(43) Pub. Date: Mar. 6, 2025

(54) ANTIMICROBIAL PEPTIDES AND METHODS OF USING SAME

(71) Applicant: Zoetis Services LLC, Parsippany, NJ (US)

(72) Inventors: Michael Kuhn, Kalamazoo, MI (US); Christopher A. Zook, Kalamazoo, MI (US); Derek James Sheehan, Galesburg, MI (US); Eric Baima, Kalamazoo, MI (US); Richard Andrew Ewin, Kalamazoo, MI (US); Hilary Phelps, Jackson, MI (US)

(21) Appl. No.: 18/746,401

(22) Filed: Jun. 18, 2024

Related U.S. Application Data
(63) Continuation of application No. 17/738,572, filed on May 6, 2022, now Pat. No. 12,054,518.
Application No. 17/738,572 is a continuation of application No. 16/769,025, filed on Jun. 2, 2020, now Pat. No. 11,352,396.
(60) Provisional application No. 62/595,725, filed on Dec. 7, 2017.

(51) Int. Cl. C07K 14/00; A61K 38/00; A61P 31/04; C07K 7/08

(52) U.S. Cl. CPC C07K 14/001; A61K 38/00; A61P 31/04; C07K 7/08

(57) ABSTRACT

Antimicrobial peptides of general formula X₀X₁X₂C X₃X₄X₅CX₆X₇X₈X₉CYX₁₀X₁₁CX₁₂X₁₃ are provided. Also provided are certain formulations containing these peptides and methods of using these peptides for treating skin infections in an animal in need thereof.

### CROSS-REFERENCE TO RELATED APPLICATIONS

This application is a continuation of U.S. application Ser. No. 17/738,572, filed on May 6, 2022, now U.S. Pat. No. 12,054,518, which is a continuation of U.S. application Ser. No. 16/769,025 filed on Jun. 2, 2020, now U.S. Pat. No. 11,352,396, which is a National Stage of PCT Application PCT/US2018/064029 filed on Dec. 5, 2018, which is a non-provisional application of U.S. Provisional Application Ser. No. 62/595,725 filed Dec. 7, 2017.

### SEQUENCE LISTING INFORMATION

The instant application contains a Sequence Listing which has been submitted electronically in XML file format and is hereby incorporated by reference in its entirety. Said XML copy, created on Nov. 19, 2024, is named ZP000218C.xml and is 152,517 bytes in size.

### FIELD OF THE INVENTION

This invention is in the field of antimicrobial peptides and uses of such peptides for treatment of infections.

### BACKGROUND

Antibiotics are chemical substances having the capacity, in a dilute solution, to kill or inhibit growth of microorganisms. Antibiotics that are sufficiently nontoxic to the host are used as chemotherapeutic agents to treat infectious diseases of humans, animals, and plants. The term was originally restricted to substances produced by microorganisms, but has been extended to include synthetic and semi-synthetic compounds of similar chemical activity.

Extensive and widespread use of antimicrobial drugs led to the emergence of resistant strains of microorganisms. These microorganisms are no longer susceptible to currently available antimicrobial drugs. In order to lower or prevent lethal infectious diseases and maintain public health, new antimicrobial agents are required.

Antimicrobial Peptides (AMPs) are an essential component of the host defense system of organisms throughout nature and offer protection from invading pathogens. They show potent antimicrobial activity against Gram-positive and Gram-negative bacteria, fungi, parasites and viruses. The smaller AMPs (usually about 15-40 amino acids) act largely by disrupting the structure or function of microbial cell membranes, they do not target single defined molecular structures. Therefore, as opposed to conventional antibiotics, they are effective regardless of the metabolic activity of bacteria. Human AMPs such as defensins and cathelicidin (LL-37) are present in leukocytes and secreted by various epithelia in skin and mucosal surfaces. In addition to their antimicrobial activity, AMPs are important effector molecules in inflammation, immune activation, and wound healing. AMPs are quite diverse in sequence and secondary structure, but share some common properties. They are usually cationic, amphipathic and exert their microbicidal effect by compromising the bacterial membrane integrity. Interaction of AMPs with the anionic membrane surface of the target microbes leads to membrane permeabilization, cell lysis and death.

### SUMMARY OF INVENTION

In the first aspect, the invention provides an amino acid sequence of 17-22 amino acids long and comprising, at its N-terminus, SEQ ID NO:12

(X₀X₁X₂CX₃X₄X₅CX₆X₇X₈X₉CYX₁₀X₁₁CX₁₂X₁₃) wherein X₀ is absent or proline; X₁ is lysine, arginine, glycine, or proline; X₂ is phenylalanine, tryptophan, or arginine; X₃ is phenylalanine, valine or tryptophan; X₄ is arginine, tyrosine or phenylalanine; X₅ is valine or alanine; X₆ is tyrosine or arginine or lysine or tryptophan; X₇ is arginine, phenylalanine, or glycine; X₈ is arginine, phenylalanine or glycine; X₉ is isoleucine, alanine, phenylalanine, tyrosine or valine; X₁₀ is arginine or histidine; X₁₁ is arginine or lysine; X₁₂ is arginine, lysine, or asparagine; X₁₃ is a 0-4 amino-acid-long polypeptide; with provisos that if X₀ is proline, then X₁ is not proline; if said amino acid sequence comprises SEQ ID NO: 13 (KWCFRVCYRGICYRRCR), or SEQ ID NO: 28 (KWCFRVCYRGICYRKCR) then X₁₃ is 1-4 amino acids long; if X₁₃ is 1 amino acid long or longer, then the N-terminal amino acid in X₁₃ is aspartic acid or glutamic acid; if the amino acid at position corresponding to position 1 of SEQ ID NO: 1 is glycine, then said glycine is not acyl- or palmitic acid—modified; if amino acid is X₁₁ lysine then X₆X₇X₈X₉ (SEQ ID NO: 14) is not RRRF (SEQ ID NO: 15); and if the amino acid is GFCWYVCYRGICYRRCN (SEQ ID NO: 16) then the C-terminal asparagine is amidated.

In certain embodiments, X₀ is absent and X₆ is arginine or lysine; and/or X₇ is arginine or lysine; X₆X₇X₈X₉ (SEQ ID NO: 14) is selected from the group consisting of YRGI (SEQ ID NO: 17), YRGV (SEQ ID NO: 18), YRGF (SEQ ID NO: 19); and/or X₁₀ is arginine; and/or X₃X₄X₅(SEQ ID NO: 20) is FRV (SEQ ID NO: 21), WYV (SEQ ID NO: 22); and/or X₁₃ is 1 amino acid long or longer, and the N-terminal amino acid in X₁₃ is aspartic acid.

In a particular set of embodiments, the amino acid sequence is 17-21 amino acids long and comprises, at its N-terminus, SEQ ID NO:12 (X₀X₁X₂C X₃X₄X₅CX₆X₇X₈X₉CYX₁₀X₁₁CX₁₂X₁₃) wherein X₀ is absent; X₁ is lysine, arginine or glycine; X₂ is phenylalanine, tryptophan, or argninine; X₃ is phenylalanine, valine or tryptophan; X₄ is tyrosine or phenylalanine; X₅ is valine or alanine; X₆ is tyrosine or arginine; X₇ is arginine or glycine; X₈ is arginine, phenylalanine or glycine; X₉ is alanine, phenylalanine, tyrosine or valine; X₁₀ is arginine or histidine; X₁₁ is arginine or lysine X₁₂ is arginine, lysine, or asparagine; X₁₃ is a 0-4 amino-acid-long polypeptide.

In a another set of embodiments according to the first aspect, the amino acid sequence is 18-21 amino acids long and comprises, at its N-terminus, SEQ ID NO: 1(KWCFRVCYRGICYRRCRD) or a peptide that differs from SEQ ID NO: 1 by one, two, three, or four amino acids, wherein the amino acids differing from the amino acids of SEQ ID NO: 1 are independently selected from the group consisting of arginine or glycine at position corresponding to position 1 of SEQ ID NO: 1; phenylalanine or arginine at position corresponding to position 2 of SEQ ID NO: 1; valine or tryptophan at position corresponding to position 4 of SEQ ID NO: 1; tyrosine at position corresponding to position 5 of SEQ ID NO: 1; arginine at position corresponding to position 8 of SEQ ID NO: 1; glycine at position corresponding to position 9 of SEQ ID NO: 1; arginine at position corresponding to position 10 of SEQ ID NO: 1; alanine, phenylalanine, or valine at position corresponding to position 11 of SEQ ID NO: 1; histidine at position corresponding to position 14 of SEQ ID NO: 1; lysine at position corresponding to position 15 of SEQ ID NO: 1; asparagine at position corresponding to position 17 of SEQ ID NO: 1.

More specifically, the amino acid sequence comprises aspartic acid at position corresponding to position 18 of SEQ ID NO: 1; and/or asparagine at position corresponding to position 17 of SEQ ID NO: 1; and/or glycine at position corresponding to position 1 of SEQ ID NO: 1; alanine at position corresponding to position 11 of SEQ ID NO: 1; and/or arginine at position corresponding to position 14 of SEQ ID NO: 1, at position corresponding to position 15 of SEQ ID NO: 1, or both.

In a set of embodiments, the amino acid sequence is selected from the group consisting of amino acid sequences comprising, at the respective N-termini, SEQ ID NO: 1, SEQ ID NO: 2 (RWCFRVCYRGICYRRCRD), SEQ ID NO: 3 (GWCFRVCYRGICYRRCRD), SEQ ID NO: 4 (KFCFRVCYRGICYRRCRD); SEQ ID NO: 5 (KWCFYVCYRGICYRRCRD), SEQ ID NO: 6 (KWCFRVCRRGICYRRCRD), SEQ ID NO: 7 (KWCFRVCYRGVCYRRCRD), SEQ ID NO: 8 (KWCFRVCYRGACYRRCRD), SEQ ID NO: 9 (KWCFRVCYRGFCYRRCRD), SQE ID NO: 10 (KWCFRVCYRGICYHRCRD), or SEQ ID NO: 11 (KWCFRVCYRGICYRRCND).

In additional embodiments, the amino acid sequence is selected from the group consisting of SEQ ID NO: 97 (KRCFRVCYRGICYRRCRD); SEQ ID NO: 98 (KWCVRVCYRGICYRRCRD), SEQ ID NO: 99 (KWCFFVCYRGICYRRCRD), SEQ ID NO: 100 (KWCFWVCYRGICYRRCRD), SEQ ID NO: 102 (KWCFRACYRGICYRRCRD), SEQ ID NO: 104 (KWCFRVCYFGICYRRCRD), SEQ ID NO: 105 (KWCFRVCYRGICYRRCRN), SEQ ID NO: 106 (KWCWRVCYRGICYRRCRD), SEQ ID NO: 107 (KWCFRVCWRGICYRRCRD), SEQ ID NO: 108 (KWCFRVCYGGICYRRCRD), SEQ ID NO: 109 (KWCFRVCYRRICYRRCRD), SEQ ID NO: 110 (KWCFRVCYRGYCYRRCRD), SEQ ID NO: 112 (KWCFRVCYRGICYRRCKD), SEQ ID NO: 113 (KWCFRVCYRGICYRRCAD), SEQ ID NO: 114 (KWCFRVCYRGICYRRCRR), SEQ ID NO: 115 (GWCFRVCYRGICYRRCND), SEQ ID NO: 116(KWCFYVCYRGICYRRCND), SEQ ID NO: 117 (GWCFYVCYRGICYRRCRD), SEQ ID NO: 32 (GWCFYVCYRGICYRRCND).

In yet additional embodiments, the amino acid sequence is selected from the group consisting of SEQ ID NO: 28, 29, 30, 31.

In the second aspect, the invention provides a multimer comprising a plurality of repeats of the amino acid sequence according to the previous aspect of the invention, wherein further, the N-terminal amino acid of said sequence is proline, and the C-terminal amino acid of said sequence is aspartic acid. Advantageously, the repeats of the amino acid sequence are joined each other directly, thereby forming D-P bonds. In certain embodiments, the plurality is between 2 and 20.

The invention also provides a method of making the amino acid sequence that is suitable for making the multimer as described in the second aspect of the invention. The method comprises synthesizing the multimer and contacting the multimer with a mild acid (e.g., formic acid) whereby D-P bonds are broken.

In a third aspect, the invention provides a method of treating infections in an animal in need thereof, comprising administering to the animal a formulation comprising the amino acid sequence according to the first aspect of the invention. In certain embodiments, the infection is a skin infection. In other embodiments, the infection is mastitis, a respiratory infection, an ear infection, urinary tract infection, or reproductive tract infection.

In certain embodiments, the animal is a companion animal, e.g., a dog, a cat, or a horse. In a particular embodiment, the animal is a dog. In certain embodiments, the formulation is formulated for a topical application. In some embodiments, the formulation is a gel, a cream, an emulsion, or a spray.

### BRIEF DESCRIPTION OF THE DRAWINGS

FIG. 1 illustrates toxicity of SEQ ID NOs: 1 and 13 in human, beagle, and rat red blood cells.

### DETAILED DESCRIPTION

### Definitions

For a better understanding of the invention, the following non-limiting definitions are provided:

“About” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater, unless about is used in reference to time intervals in weeks where “about 3 weeks,” is 17 to 25 days, and about 2 to about 4 weeks is 10 to 40 days.

“Emulsion” means a composition of two immiscible liquids in which small droplets of one liquid are suspended in a continuous phase of the other liquid.

“Parenteral administration” refers to the introduction of a substance, such as a vaccine, into a subject's body through or by way of a route that does not include the digestive tract.

Parenteral administration includes subcutaneous, intramuscular, transcutaneous, intradermal, intraperitoneal, intraocular, and intravenous administration.

“Position [in a sequence of interest] corresponding to” a certain position of a reference sequence is determined by aligning the reference sequence and the sequence of interest in such a way that the cysteine residues of the sequence of interest and the reference sequence are matched to each other, and then determining the position in the sequence of interest that matcher the desired position in the reference sequence.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

“Therapeutically effective amount” refers to an amount of the amino acid sequence and/or the formulation containing same that would induce a response in a subject receiving the amino acid or formulation which is adequate to prevent or reduce signs or symptoms of infection.

“Treating” refers to preventing a disorder, condition, or disease, including, without limitations, infections, to which such term applies, or to preventing or reducing one or more symptoms of such disorder, condition, or disease.

“Treatment” refers to the act of “treating” as defined above.

### Peptides

Generally, the invention provides an amino acid sequence of 17-22 amino acids long and comprising, at its N-terminus, SEQ ID NO:12 (X₀X₁X₂C X₃X₄X₅CX₆X₇X₈X₉CYX₁₀X₁₁CX₁₂X₁₃) wherein
X₀ is absent or proline;
X₁ is lysine, arginine, glycine, or proline;
X₂ is phenylalanine, tryptophan, or arginine;
X₃ is phenylalanine, valine or tryptophan;
X₄ is arginine, tyrosine or phenylalanine;
X₅ is valine or alanine;
X₆ is tyrosine or arginine;
X₇ is arginine, phenylalanine, or glycine;
X₈ is arginine, phenylalanine or glycine;
X₉ is isoleucine, alanine, phenylalanine, tyrosine or valine;
X₁₀ is arginine or histidine;
X₁₁ is arginine or lysine;
X₁₂ is arginine, lysine, or asparagine;
X₁₃ is a 0-4 amino-acid-long polypeptide;
   with provisos that if X₀ is proline, then X₁ is not proline; if said amino acid sequence comprises SEQ ID NO: 13 (KWCFRVCYRGICYRRCR), or SEQ ID NO: 28 (KWCFRVCYRGICYRKCR) then X₁₃ is 1-4 amino acids long; if X₁₃ is 1 amino acid long or longer, then the N-terminal amino acid in X₁₃ is aspartic acid or glutamic acid; if the amino acid at position corresponding to position 1 of SEQ ID NO: 1 is glycine, then said glycine is not acyl-or palmitic acid-modified; if amino acid is X₁₁ lysine then X₆X₇X₈X₉ (SEQ ID NO: 14) is not RRRF (SEQ ID NO: 15); and if the amino acid is GFCWYVCYRGICYRRCN (SQE ID NO: 16) then the C-terminal asparagine is amidated.

In certain embodiments, X₀ is absent and X₆ is arginine or lysine; and/or X₇ is arginine or lysine; X₆X₇X₈X₉ (SEQ ID NO: 14) is selected from the group consisting of YRGI (SEQ ID NO: 17), YRGV (SEQ ID NO: 18), YRGF (SEQ ID NO: 19); and/or X₁₀ is arginine; and/or X₃X₄X₅ (SEQ ID NO: 20) is FRV (SEQ ID NO: 21), WYV (SEQ ID NO: 22); and/or X₁₃ is 1 amino acid long or longer (e.g., 1, 2, 3, or 4 amino acids long), and the N-terminal amino acid in X₁₃ is aspartic acid.

In a particular set of embodiments according to the first aspect, the amino acid sequence is 18-21 amino acids long and comprises, at its N-terminus, SEQ ID NO: 1(KWCFRVCYRGICYRRCRD) or a peptide that differs from SEQ ID NO: 1 by one, two, three, or four amino acids, wherein the amino acids differing from the amino acids of SEQ ID NO: 1 are independently selected from the group consisting of arginine or glycine at position corresponding to position 1 of SEQ ID NO: 1; phenylalanine or argninine at position corresponding to position 2 of SEQ ID NO: 1; valine or tryptophan at position corresponding to position 4 of SEQ ID NO: 1; tyrosine at position corresponding to position 5 of SEQ ID NO: 1; arginine at position corresponding to position 8 of SEQ ID NO: 1; glycine at position corresponding to position 9 of SEQ ID NO: 1; arginine at position corresponding to position 10 of SEQ ID NO: 1; alanine, phenylalanine, or valine at position corresponding to position 11 of SEQ ID NO: 1; histidine at position corresponding to position 14 of SEQ ID NO: 1; lysine at position corresponding to position 15 of SEQ ID NO: 1; asparagine at position corresponding to position 17 of SEQ ID NO: 1.

In different embodiments, the amino acid sequence differs from SEQ ID NO: 1 by one, two, or three amino acids.

In certain embodiments, the amino acid sequence comprises aspartic acid at position corresponding to position 18 of SEQ ID NO: 1; and/or asparagine at position corresponding to position 17 of SEQ ID NO: 1; and/or glycine at position corresponding to position 1 of SEQ ID NO: 1; alanine at position corresponding to position 11 of SEQ ID NO: 1; and/or arginine at position corresponding to position 14 of SEQ ID NO: 1, at position corresponding to position 15 of SEQ ID NO: 1, or both.

In a set of embodiments, the amino acid sequence is selected from the group consisting of amino acid sequences comprising, at the respective N-termini, SEQ ID NO: 1, SEQ ID NO: 2 (RWCFRVCYRGICYRRCRD), SEQ ID NO: 3 (GWCFRVCYRGICYRRCRD), SEQ ID NO: 4 (KFCFRVCYRGICYRRCRD); SEQ ID NO: 5 (KWCFYVCYRGICYRRCRD), SEQ ID NO: 6 (KWCFRVCRRGICYRRCRD), SEQ ID NO: 7 (KWCFRVCYRGVCYRRCRD), SEQ ID NO: 8 (KWCFRVCYRGACYRRCRD), SEQ ID NO: 9 (KWCFRVCYRGFCYRRCRD), SQE ID NO: 10 (KWCFRVCYRGICYHRCRD), or SEQ ID NO: 11 (KWCFRVCYRGICYRRCND).

Additional amino acid sequences may be found among SEQ ID NO: 97 (KRCFRVCYRGICYRRCRD); SEQ ID NO: 98 (KWCVRVCYRGICYRRCRD), SEQ ID NO: 99 (KWCFFVCYRGICYRRCRD), SEQ ID NO: 100 (KWCFWVCYRGICYRRCRD), SEQ ID NO: 101 (KWCFRVYCYRGICYRRCRD), SEQ ID NO: 102 (KWCFRACYRGICYRRCRD), SEQ ID NO: 103 (KWCFRVCKRGICYRRCRD), SEQ ID NO: 104 (KWCFRVCYFGICYRRCRD), SEQ ID NO: 105 (KWCFRVCYRGICYRRCRN), SEQ ID NO: 106 (KWCWRVCYRGICYRRCRD), SEQ ID NO: 107 (KWCFRVCWRGICYRRCRD), SEQ ID NO: 108 (KWCFRVCYGGICYRRCRD), SEQ ID NO: 109 (KWCFRVCYRRICYRRCRD), SEQ ID NO: 110(KWCFRVCYRGYCYRRCRD), SEQ ID NO: 111 (KWCFRVCYRGICRYRRCRD), SEQ ID NO: 112 (KWCFRVCYRGICYRRCKD), SEQ ID NO: 113 (KWCFRVCYRGICYRRCAD), SEQ ID NO: 114 (KWCFRVCYRGICYRRCRR), SEQ ID NO: 115 (GWCFRVCYRGICYRRCND), SEQ ID NO: 116 (KWCFYVCYRGICYRRCND), SEQ ID NO: 117 (GWCFYVCYRGICYRRCRD), SEQ ID NO: 32 (GWCFYVCYRGICYRRCND).

Thus, the amino acid sequence may be selected from the group consisting of SEQ ID NO: 97 (KRCFRVCYRGICYRRCRD); SEQ ID NO: 98 (KWCVRVCYRGICYRRCRD), SEQ ID NO: 99 (KWCFFVCYRGICYRRCRD), SEQ ID NO: 100 (KWCFWVCYRGICYRRCRD), SEQ ID NO: 102 (KWCFRACYRGICYRRCRD), SEQ ID NO: 104 (KWCFRVCYFGICYRRCRD), SEQ ID NO: 105 (KWCFRVCYRGICYRRCRN), SEQ ID NO: 106 (KWCWRVCYRGICYRRCRD), SEQ ID NO: 107 (KWCFRVCWRGICYRRCRD), SEQ ID NO: 108 (KWCFRVCYGGICYRRCRD), SEQ ID NO: 109 (KWCFRVCYRRICYRRCRD), SEQ ID NO: 110 (KWCFRVCYRGYCYRRCRD), SEQ ID NO: 112 (KWCFRVCYRGICYRRCKD), SEQ ID NO: 113 (KWCFRVCYRGICYRRCAD), SEQ ID NO: 114 (KWCFRVCYRGICYRRCRR), SEQ ID NO: 115 (GWCFRVCYRGICYRRCND), SEQ ID NO: 116 (KWCFYVCYRGICYRRCND), SEQ ID NO: 117 (GWCFYVCYRGICYRRCRD), SEQ ID NO: 32 (GWCFYVCYRGICYRRCND).

The peptides according to the invention can be manufactured by methods that are well-known in the art, including, without limitations, solid-phase peptide synthesis. The peptides may also be synthesized using bioengineering techniques (e.g., fermentation) in fungal, bacterial or eukaryotic systems.

In certain embodiments, where the N-terminal amino acid of the antimicrobial peptide is proline, and the C-terminal amino acid is aspartic acid, the method of manufacturing the anti-microbial peptide may entail synthesizing a multimer of the antimicrobial peptide. In different embodiments, the number of monomers in the multimer may be 1 to about 20, e.g., about 5,about 10, or about 15. Conveniently, the monomers of the antimicrobial peptide would be linked via a peptide bond between the C-terminal aspartic acid of an upstream monomer and the N-terminal proline of the downstream monomer (D-P bond). This D-P bond can conveniently be cleaved via mild acid (e.g., formic or citric acid) hydrolysis. Thus, a molecule encompassed by such description as, for example, (SEQ ID NO: 29) n or (SEQ ID NO: 31) n, wherein n is an integer between 1 and 20, may be used in the compositions and methods of the invention.

### Formulations

The peptides according to the embodiments above may be formulated for delivery to the target site (i.e., the site that is infected or the site that is in danger of being infected due to a wound, irritation, to the like). Without limitation, the sites include skin, eyes, ears, mammary gland, reproductive tract, urinary bladder, nasal and oral cavities. The composition comprising the peptides of the instant invention is formulated depending on the site of interest.

Also provided are compositions that can be prepared by mixing one or more antimicrobial peptides described herein, with pharmaceutically acceptable carriers, excipients, binders, diluents or the like, to treat or ameliorate a variety of bacterial infections. A therapeutically effective dose or amount refers to that amount of one or more compounds described herein sufficient to result in amelioration of symptoms of the infection. The pharmaceutical compositions of the instant invention can be manufactured by methods well known in the art such as conventional granulating, mixing, dissolving, encapsulating, lyophilizing, or emulsifying processes, among others. The compositions can be in the form of, for example, granules, powders, tablets, capsule syrup, suppositories, injections, emulsions, elixirs, suspensions or solutions. The instant compositions can be formulated for various routes of administration, for example, by oral administration, by topical administration, by transmucosal administration, by rectal administration, or subcutaneous administration as well as intrathecal, intravenous, intramammary, intramuscular, intraperitoneal, intranasal, intraocular or intraventricular injection. The compound or compounds of the instant invention can also be administered in a local fashion, such as injection as a sustained release formulation. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive or excipient such as a starch or other additive. Suitable additives or excipients are sucrose, lactose, cellulose sugar, mannitol, maltitol, dextran, sorbitol, starch, agar, alginates, chitins, chitosans, pectins, tragacanth gum, gum arabic, gelatins, collagens, casein, albumin, synthetic or semi-synthetic polymers or glycerides, methyl cellulose, hydroxypropylmethyl-cellulose, and/or polyvinylpyrrolidone. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Additionally, dyestuffs or pigments can be added for identification. Tablets and pills can be further treated with suitable coating materials known in the art.

Liquid dosage forms for oral administration can be in the form of pharmaceutically acceptable emulsions, syrups, elixirs, suspensions, slurries and solutions, which can contain an inactive diluent, such as water. Pharmaceutical formulations can be prepared as liquid suspensions or solutions using a sterile liquid, such as, but not limited to, an oil, water, an alcohol, and combinations of these. Pharmaceutically suitable surfactants, suspending agents, emulsifying agents, can be added for oral or parenteral administration.

As noted above, suspensions can include oils. Such oils include peanut oil, sesame oil, cottonseed oil, corn oil, olive oil and mixtures of oils. Suspension preparation can also contain esters of fatty acids such as ethyl oleate, isopropyl myristate, fatty acid glycerides and acetylated fatty acid glycerides. Suspension formulations can include alcohols, such as, but not limited to, ethanol, isopropyl alcohol, hexadecyl alcohol, glycerol and propylene glycol. Ethers, such as but not limited to, poly (ethyleneglycol), petroleum hydrocarbons such as mineral oil and petrolatum; and water can also be used in suspension formulations.

For certain routes of administration, the pharmaceutical formulations can be a spray or aerosol containing and appropriate solvents and optionally other compounds such as, but not limited to, stabilizers, antimicrobial agents, antioxidants, pH modifiers, surfactants, bioavailability modifiers and combinations of these. A propellant for an aerosol formulation can include compressed air, nitrogen, carbon dioxide, or a hydrocarbon based low boiling solvent. The compound or compounds of the instant invention are conveniently delivered in the form of an aerosol spray presentation from a nebulizer or the like.

Injectable dosage forms generally include aqueous suspensions or oil suspensions which can be prepared using a suitable dispersant or wetting agent and a suspending agent. Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Generally, the oil or fatty acid is non-volatile, including natural or synthetic oils, fatty acids, mono-, di-or tri-glycerides.

For injection, the pharmaceutical formulation can be a powder suitable for reconstitution with an appropriate solution as described above. Examples of these include freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

For rectal administration, the pharmaceutical formulations can be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils can also be employed in the preparation of formulations of the soft gelatin type and suppositories. Water, saline, aqueous dextrose and related sugar solutions, and glycerols can be employed in the preparation of suspension formulations which can also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carries are generally known to those skilled in the art and are thus included in the instant invention. Such excipients and carriers are described, for example, in “Remington's Pharmaceutical Sciences”, Mack Pub. Co., New Jersey (1991).

The formulations of the invention can be designed to be short-acting, fast-releasing, long-acting, and sustained-releasing. Thus, the pharmaceutical formulations can also be formulated for controlled release or for slow release.

The instant compositions can also comprise, for example, micelles or liposomes, or some other encapsulated form, or can be administered in an extended release form to provide a prolonged storage and/or delivery effect. Therefore, the pharmaceutical formulations can be compressed into pellets or cylinders and implanted intramuscularly or subcutaneously as depot injections or as implants such as stents. Such implants can employ known materials such as silicones and biodegradable polymers.

The composition may also contain anti-pruritic medications, including, without limitations, oclatinib and salts thereof (e.g., APOQUEL® and anti-IL-31 antibodies (e.g., CYTOPOINT™).

The composition can also comprise a steroid or an anti-fungal medicine. Suitable steroids include, without limitations, Betamethasone, triamcinolone acetonide, hydrocortisone aceponate, hydrocortisone, triamcinolone, methylprednisolone acetate, and the like. Suitable anti-fungal medicines include, without limitations chlotrimazole, econazole, itraconazole, ketoconazole, miconazole.

The compositions can contain, for example, from about 0.1% by weight, to about 90% or more by weight, of the antimicrobial peptide, depending on the method of administration. Where the compositions comprise dosage units, each unit can contain, for example, from about 0.5 mg to about 10 mg per dose of the antimicrobial peptide. For example, one dose of the composition may contain about 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg. The composition may contain about 1 to about 5 mg of the antimicrobial peptide per dose, or about 1.5 to about 5 mg of the antimicrobial peptide per dose, or about 2.5 mg to about 7.5 mg per dose, or about 1.5 mg to about 2.5 mg per dose, depending on the severity of the wound and the size of the animal.

### Methods

In yet another aspect, the invention also provides methods of treating or preventing a bacterial infection in a subject, comprising administering an effective amount of one or more compounds described herein to the subject. Suitable subjects that can be treated include dogs, cats, horses, cattle, sheep, pigs, poultry, primates (e.g., rhesus monkeys and cynomolgus (also known as crab-eating or long-tailed) monkeys, marmosets, tamarinds, chimpanzees, macaques), rabbits, and rodents (rats, mice, guinea pigs and the like). In certain embodiment, the subject is a dog, and the antimicrobial peptide of the invention is delivered topically, intranasally, intraocularly, or intraaurally. The antimicrobial peptide may be delivered in a form of drops, spray, cream, gel, ointment and the like.

Infections that can be treated with the described compounds include external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, burns, antibacterial prophylaxis of surgery, and antibacterial prophylaxis in immunosuppressed subjects, such as patients receiving cancer chemotherapy, or organ transplant patients. These infections can be treated in hospital or community settings via various routes of administration as described herein.

The compounds or compositions described herein can also be used prophylactically. Accordingly, one or more of the present compounds or compositions can be administered to a subject deemed to be at risk for developing a microbial infection. Subjects at risk for developing a microbial infection include individuals who have been exposed to a particular microorganism, such as a pathogenic bacterial species; individuals having a compromised immune system, or subjects that are particularly vulnerable to the infections due to compromised natural defenses (e.g., where the skin is compromised due to burns or cuts).

The antimicrobial peptides described herein can be used for the treatment or prevention of infectious disorders caused by a variety of bacterial organisms, including infections by pathogenic bacterial species. Non-limiting examples of bacterial infection include Gram positive and Gram negative aerobic and anaerobic bacteria, such as Staphylococci, e.g., S. aureus; Enterococci, e.g., E. faecalis; Streptococci, e.g., S. pyogenes and S. pneumoniae; Escherichiaspecies, e.g., E. coli, including enterotoxigenic, enteropathogenic, enteroinvasive, enterohemorrhagic and enteroaggregative E. coli strains; Propionibacterium strains, e.g., P. acnes; Haemophilus, e.g., H. influenza; Moraxella, e.g., M. catarrhalis. Other examples include Mycobacteria, e.g., M. tuberculosis, M. avian-intracellulare, M. kansasii, M. bovis, M. africanum, M. genavense, M. leprae, M. xenopi, M. simiae, M. scrofulaceum, M. malmoense, M. celatum, M. abscessus, M. chelonae, M. szulgai, M. gordonae, M. haemophilusrtuni and M. marinum; Corynebacteria, e.g., C. diphtheriae; Pseudomonas species, e.g., P. aeruginosa; Borrelia species, e.g., B. burgdorferi; Listeria species, e.g., L. monocytogenes; Bacillus species, e.g., B. cereus; Bordetella species, e.g., B. bronchiseptica; Klebsiella species, Clostridium species, e.g., C. perfringens, C. tetani; Chlamydia species, e.g., C. psittaci,; Rickettsia species, e.g., R. rickettsii and R. prowazekii; Salmonella species, e.g., S. typhimurium; Yersinia species, e.g., Y. enterocolitica and Y. pseudotuberculosis; Klebsiella species, e.g., K. pneumoniae; and Mycoplasma, e.g., M. pneumonia, Actinobacillus species, H. parasuis; and Trueperella pyogenes.

In certain aspects the bacteria are selected fromStaphylococci, e.g, S. pseudintermedius, S. aureus, S. schleiferi, S. chromogenes, S. simulans, S. xylosus. The bacteria may also be selected from Streptococci, e.g., S. uberis, S. agalactiae, S. dysgalactiae, S. suis. Further, the bacteria of family Pasteurellaceae are suitable for treatment with the compositions described herein. Suitable Pasteurellaceae bacteria include M. haemolytica, P. multocida, H. somni, Escherichia species, e.g., E. coli, and Klebsiella species.

In certain embodiments, the bacteria are S. pseudintermedius and/or P. aeruginosa.

The compositions described herein may be administered in different frequency regiments. For example, suitable regimens include 4 times daily to once every week, e.g., three times daily, twice daily, once daily, every two days, every three days, twice per week, every five days and so on. Similarly, the inventions described herein may be administered in different duration regimens, e.g., in a single administration, for two days, for three days, for four days, for a week, for two weeks, for a month, for six weeks, and so on. The duration, the frequency and the amount of the antimicrobial peptide per dose, as well as the species and the state of the wound and/or state of the infection, may be considered together in determining the proper dose-time-frequency regimen for administration of the antimicrobial compositions claimed herein.

The following examples are presented as illustrative embodiments, but should not be taken as limiting the scope of the invention. Many changes, variations, modifications, and other uses and applications of this invention will be apparent to those skilled in the art.

### EXAMPLES

### Example 1. Antimicrobial activity and safety in vitro

Peptides according to SEQ ID NOs as listed in Table 1 were prepared by a commercial manufacturer (CS Bio, Menlo Park, California) using solid phase synthesis. Antimicrobial activity was assessed by determining the Minimal Inhibitory Concentration (MIC) against S. aureus andE. coli. Briefly, Microbroth MICs were performed using CLSI methodology (VET01-S2). For S. aureus and E. coli ATCC strains, TSA with 5% lysed horse blood agar was used for overnight culturing at 37° C. ambient air. A 0.5 mM stock for each peptide was made with cell culture water, 0.01% acetic acid and serially diluted and spotted (10 μL) in a 96-well plate for in assay dose titration concentration of 50 μM to 0.05 pM. 0.5 McFarland Standard of each strain was diluted 1:250 in Mueller-Hinton Broth (MHB). 90 μL of culture suspension was then added upon drug in the 96-well plate for overnight incubation for 18-20 hours. The MIC was determined visually at the first well of no visible growth at the corresponding concentration.

The results of these experiments are provided in Table 1.

TABLE 1

| | | S. aureus | E. coli |
|---|---|---|---|
| | | ATCC 29213 | ATCC 25922 |
| SEQ | Structure | μM | μM |
| SEQ ID NO: 23 | KFCVYVCYRGICYRRCK | 1.6 | 0.4 |
| SEQ ID NO: 24 | KWCFRVCYRGVCYRRCR | 1.6 | 0.4 |
| SEQ ID NO: 1 | KWCFRVCYRGICYRRCRD | 1.6 | 0.4 |
| SEQ ID NO: 13 | KWCFRVCYRGICYRRCR | 3.1 | 0.8 |
| SEQ ID NO: 91 | GFCWYVCYRGFCYRRCN | 3.1 | 0.8 |
| SEQ ID NO: 92 | RGGRLCYCRRRFCVCVGR | 3.1 | 3.1 |
| SEQ ID NO: 93 | RRWCFRVCYRGFCYRKCR | 3.1 | 1.6 |
| SEQ ID NO: 28 | KWCFRVCYRGICYRKCR | 3.1 | 1.6 |
| SEQ ID NO: 94 | GFCWYVCRRRFCYRRCN | 3.1 | 0.4 |
| SEQ ID NO: 25 | KWCFRVCRRRFCYRRCR | 3.1 | 0.8 |
| SEQ ID NO: 26 | GFCWYVCYRGICYRRCN-NH2 | 3.1 | 0.8 |
| SEQ ID NO: 27 | GFCWYVCYRGFCYRRCN-NH2 | 3.1 | 0.8 |
| SEQ ID NO: 95 | GFCWYVCRRRFCYRRCN | 6.2 | 0.4 |
| SEQ ID NO: 33 | PGFCWYVCRRRFCYRRCN | 6.2 | 0.4 |
| SEQ ID NO: 34 | PFCWYVCRRRFCYRRCN | 6.2 | 0.4 |
| SEQ ID NO: 35 | GFCWYVCRRRFCHRRCN | 6.2 | 0.4 |
| SEQ ID NO: 36 | GVCVYVCRRRFCYRRCN | 6.2 | 0.4 |
| SEQ ID NO: 37 | GVCVYVCRRRFCYRRCN | 6.2 | 0.4 |
| SEQ ID NO: 38 | GFCWYVCRRRFCYRRCN-NH2 | 6.2 | 0.4 |
| SEQ ID NO: 39 | PGFCWYVCRRRFCYRRCND | 6.2 | 0.4 |
| SEQ ID NO: 40 | GFCWYVCRRRHCYRRCN | 6.2 | 0.8 |
| SEQ ID NO: 41 | KFCVYVCRRRFCYRRCK | 6.2 | 0.8 |
| SEQ ID NO: 42 | GHCWYVCRRRFCYRRCN | 6.2 | 0.4 |
| SEQ ID NO: 43 | GFCWYVCRRRFCYRRCS | 6.2 | 0.4 |
| SEQ ID NO: 44 | *GFCWYVCYRGICYRRCN-NH2 | 6.2 | 0.4 |
| SEQ ID NO: 45 | *GFCWYVCYRGFCYRRCN-NH2 | 6.2 | 0.8 |
| SEQ ID NO: 16 | GFCWYVCYRGICYRRCN | 6.25 | 0.4 |
| SEQ ID NO: 46 | GFCWYVCYRGFCYRRCN | 6.25 | 0.4 |
| SEQ ID NO: 47 | KFCWRVCRRRFCRRRCN | 12.5 | 0.8 |
| SEQ ID NO: 48 | GFCWYVCRRGFCYRRCN | 12.5 | 0.4 |
| SEQ ID NO: 49 | KWCFRVCRNGVCYRRCR | 12.5 | 0.2 |
| SEQ ID NO: 50 | GFCWYTCRRRFCYRRCN | 12.5 | 0.4 |
| SEQ ID NO: 51 | GFCWYVCYRGFCHRRCN | 25 | 6.2 |
| SEQ ID NO: 52 | GFCWYVCRRRFCHRRCN | 25 | 0.8 |
| SEQ ID NO: 53 | GFCWYVCRRRFCYRRCN | 25 | 50 |
| SEQ ID NO: 54 | KFCWNVCRRRFCHRRCK | 25 | 0.4 |
| SEQ ID NO: 55 | GFCWYVCRRGICYRRCN | 25 | 0.8 |
| SEQ ID NO: 56 | GFCWYVCRRGICYRRCN | 25 | 0.8 |
| SEQ ID NO: 57 | GFCWNVCRRRFCRRRCN | 25 | 0.4 |
| SEQ ID NO: 58 | KFCVNVCYRGICHRRCK | 25 | 0.2 |
| SEQ ID NO: 59 | KFCVNVCRRRFCHRRCK | 25 | 0.8 |
| SEQ ID NO: 60 | KFCVNVCRRRFCRRRCK | 25 | 0.4 |
| SEQ ID NO: 61 | GFCWYVCYRGFCYQQCN | 25 | 6.2 |
| SEQ ID NO: 62 | GFCWYVCYRGFCYDDCN | 25 | 50 |
| SEQ ID NO: 63 | GFCWYVCPKGYCYRRCN | 50 | 50 |
| SEQ ID NO: 64 | GFCWYVCKNGFCYRRCN | 50 | 3.1 |
| SEQ ID NO: 65 | RKGCKCKNGFCVCR-NH2 | 50 | 6.25 |
| SEQ ID NO: 66 | GFCWNVCRRRFCHRRCN | 50 | 3.1 |
| SEQ ID NO: 67 | GFCWNVCRRRFCHRRCN | 50 | 1.6 |
| SEQ ID NO: 68 | GFCWNVCYRGICHRRCN | 50 | 1.6 |
| SEQ ID NO: 69 | KVCVNVCKQGICRKRCK | 50 | 50 |
| SEQ ID NO: 70 | GCWYVCRNGVCYRRCN | 50 | 25 |
| SEQ ID NO: 71 | GFCWYVCRNGVCYRRCN | 50 | 3.1 |
| SEQ ID NO: 72 | GFCWNVCYRGFCHRRCN | 50 | 3.1 |
| SEQ ID NO: 73 | GFCWYVCYRGFCYHHCN | 50 | 3.1 |
| SEQ ID NO: 74 | NVCVVRCRRGFCNRRCK | 50 | 12.5 |
| SEQ ID NO: 75 | KCVRVCRRRACRRRCK | 50 | 50 |
| SEQ ID NO: 76 | KCVRVCRRGFCNRRCK | 50 | 50 |
| SEQ ID NO: 77 | GFCWYVCKNGYCYRRCN | 50 | 3.1 |
| SEQ ID NO: 78 | GFCWYVCRNGYCYRRCN | 50 | 3.1 |
| SEQ ID NO: 79 | GFCWYVCRNGYCHRRCN | 50 | 6.2 |
| SEQ ID NO: 80 | GFCWYVCYRGICHRRCN | 50 | 1.6 |
| SEQ ID NO: 81 | KCVRVCRRGFCNRRCK | 50 | 50 |
| SEQ ID NO: 82 | GFCRYVCYRGICRRRCN | 50 | 6.2 |
| SEQ ID NO: 83 | KFCVNVCRNGICRRRCK | 50 | 1.6 |
| SEQ ID NO: 84 | GVCVNVCRRRFCHRRCN | 50 | 3.1 |
| SEQ ID NO: 85 | KKVCVNVCKQGICHKRCK | 50 | 50 |
| SEQ ID NO: 86 | KKVCVNVCRQGICHRRCK | 50 | 50 |
| SEQ ID NO: 87 | GFCRYVCRRGICRRRCN | 50 | 12.5 |
| SEQ ID NO: 88 | KFCVNVCYRGICRRRCK | 50 | 0.4 |
| SEQ ID NO: 89 | KCRRVCRRGFCYVVCN | 50 | 25 |
| SEQ ID NO: 90 | GHCHHVCRRRHCHRRCN | 50 | 50 |

*refers to N-acetylation
**refers to N-palmitic acid modification

SEQ ID NO: 1 was selected for further research. Toxicity of SEQ ID NO: 1 to eukaryotic blood cells was compared to that of tachyplesin (SEQ ID NO: 13). A standard, well referenced red blood cell hemolysis assay was employed against multiple species to test the lysis potential of the peptides. Red blood cells (RBCs) were prepared and isolated by several centrifuge and wash steps to remove the plasma fraction. A dose titration (50 μM to 0.05 μM) of test peptides and control peptide melittin were spotted from 50 mM stocks in a 384 well plate. Prepared RBCs were incubated with peptide for one hour at 37° C. Percent hemolysis was measured by optical density at 405 nm and utilizing 1% TritonX100 as hundred percent effect (HPE) and phosphate buffer alone as zero percent effect (ZPE).

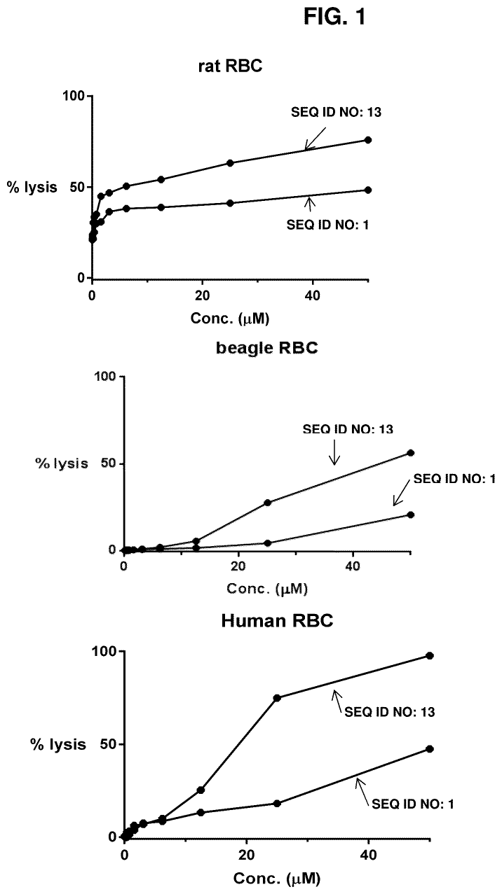

The inventors have surprisingly discovered that SEQ ID NO: 1 had not only improved anti-microbial activity but also decreased toxicity to red blood cells. The results of the experiments using human, beagle, and rat red blood cells are illustrated in FIG. 1. Briefly, SEQ ID NO: 1 was 2-4 times less toxic to human, beagle or rat red blood cells than SEQ ID NO: 13.In mouse or bovine red blood cells, the differences were negligible.

Additional derivatives of SEQ ID NO: 1 have been synthesized by solid-phase synthesis as described above. Antimicrobial activity was assessed by determining MICs against S. aureus and E. coli, as described above. The results of these experiments are provided in Table 2.

TABLE 2

| | | S. aureus | E. coli 2 |
|---|---|---|---|
| | | ATCC 29213 | ATCC 5922 |
| SEQ ID | Sequence | μM | μM |
| SEQ ID NO: 3 | GWCFRVCYRGICYRRCRD | 6.2 | 3.1 |
| SEQ ID NO: 4 | KFCFRVCYRGICYRRCRD | 3.1 | 1.6 |
| SEQ ID NO: 5 | KWCFYVCYRGICYRRCRD | 6.2 | 1.6 |
| SEQ ID NO: 2 | RWCFRVCYRGICYRRCRD | 12.5 | 0.8 |
| SEQ ID NO: 6 | KWCFRVCRRGICYRRCRD | 12.5 | 3.1 |
| SEQ ID NO: 96 | KWCFRVCYGRICYRRCRD | 3.1 | 1.6 |
| SEQ ID NO: 7 | KWCFRVCYRGVCYRRCRD | 6.2 | 1.6 |
| SEQ ID NO: 8 | KWCFRVCYRGACYRRCRD | 3.1 | 1.6 |
| SEQ ID NO: 9 | KWCFRVCYRGFCYRRCRD | 6.2 | 3.1 |
| SEQ ID NO: 10 | KWCFRVCYRGICYHRCRD | 6.2 | 1.6 |
| SEQ ID NO: 11 | KWCFRVCYRGICYRRCND | 6.2 | 0.8 |
| SEQ ID NO: 1 | KWCFRVCYRGICYRRCRD | 3.1 | 0.8 |

Antimicrobial activity of the peptides listed in Table 2 against different strains of MSSP (Methicillin-Susceptible Staphylococcus pseudintermedius) and MRSP (Methicillin-Resistant Staphylococcus pseudintermedius) was further assessed. The results are in Tables 3 and 4, respectively.

TABLE 3
MIC against selected strains of MSSP

| SEQ ID | 49051 | 71990 | 72036 | 72191 | 76986 | 77378 | 78032 | 81923 | 84250 | 84658 | 86000 | 86001 |
|---|---|---|---|---|---|---|---|---|---|---|---|---|
| 3 | 1.6 | 1.6 | 1.6 | 1.6 | 1.6 | 0.4 | 1.6 | 1.6 | 1.6 | 1.6 | 1.6 | 0.8 |
| 4 | 1.6 | 1.6 | 0.8 | 0.8 | 0.8 | 0.4 | 0.8 | 1.6 | 1.6 | 1.6 | 1.6 | 0.8 |
| 5 | 1.6 | 1.6 | 1.6 | 1.6 | 1.6 | 0.8 | 1.6 | 1.6 | 1.6 | 1.6 | 1.6 | 1.6 |
| 2 | 1.6 | 1.6 | 1.6 | 1.6 | 1.6 | 1.6 | 1.6 | 3.1 | 1.6 | 3.1 | 1.6 | 1.6 |
| 6 | 3.1 | 3.1 | 3.1 | 3.1 | 3.1 | 1.6 | 3.1 | 3.1 | 3.1 | 3.1 | 3.1 | 3.1 |
| 96 | 1.6 | 1.6 | 1.6 | 1.6 | 1.6 | 0.8 | 1.6 | 1.6 | 1.6 | 1.6 | 1.6 | 1.6 |
| 7 | 1.6 | 1.6 | 1.6 | 1.6 | 1.6 | 0.8 | 1.6 | 1.6 | 1.6 | 1.6 | 1.6 | 1.6 |
| 8 | 1.6 | 1.6 | 1.6 | 1.6 | 1.6 | 0.8 | 1.6 | 3.1 | 1.6 | 1.6 | 1.6 | 1.6 |
| 9 | 1.6 | 1.6 | 0.8 | 1.6 | 0.8 | 0.4 | 0.8 | 1.6 | 1.6 | 1.6 | 0.8 | 0.8 |
| 10 | 1.6 | 1.6 | 1.6 | 1.6 | 1.6 | 0.8 | 1.6 | 1.6 | 1.6 | 1.6 | 1.6 | 1.6 |
| 11 | 0.8 | 1.6 | 0.8 | 0.8 | 0.8 | 0.4 | 0.8 | 1.6 | 0.8 | 0.8 | 0.4 | 0.8 |
| 1 | 1.6 | 1.6 | 1.6 | 1.6 | 1.6 | 1.6 | 1.6 | 1.6 | 1.6 | 1.6 | 1.6 | 1.6 |

TABLE 4
MIC against selected strains of MRSP

| SEQ ID | 71994 | 72035 | 72192 | 76923 | 79882 | 80729 | 81926 | 86002 | 87655 | 88493 |
|---|---|---|---|---|---|---|---|---|---|---|
| 3 | 1.6 | 1.6 | 1.6 | 1.6 | 0.8 | 1.6 | 1.6 | 1.6 | 1.6 | 1.6 |
| 4 | 1.6 | 1.6 | 1.6 | 0.8 | 0.8 | 0.8 | 0.8 | 1.6 | 1.6 | 1.6 |
| 5 | 1.6 | 1.6 | 1.6 | 1.6 | 1.6 | 1.6 | 1.6 | 1.6 | 1.6 | 1.6 |
| 2 | 1.6 | 1.6 | 1.6 | 1.6 | 0.8 | 1.6 | 1.6 | 1.6 | 1.6 | 1.6 |
| 6 | 3.1 | 3.1 | 3.1 | 3.1 | 3.1 | 3.1 | 3.1 | 3.1 | 3.1 | 3.1 |
| 96 | 1.6 | 1.6 | 1.6 | 1.6 | 1.6 | 1.6 | 1.6 | 1.6 | 1.6 | 1.6 |
| 7 | 1.6 | 1.6 | 1.6 | 1.6 | 1.6 | 1.6 | 1.6 | 1.6 | 1.6 | 1.6 |
| 8 | 1.6 | 1.6 | 1.6 | 1.6 | 1.6 | 1.6 | 1.6 | 3.1 | 1.6 | 1.6 |
| 9 | 1.6 | 1.6 | 1.6 | 1.6 | 0.8 | 1.6 | 1.6 | 1.6 | 1.6 | 1.6 |
| 10 | 1.6 | 1.6 | 1.6 | 1.6 | 0.8 | 1.6 | 1.6 | 1.6 | 1.6 | 1.6 |
| 11 | 1.6 | 1.6 | 0.8 | 0.8 | 0.8 | 0.8 | 0.8 | 0.8 | 0.8 | 1.6 |
| 1 | 1.6 | 1.6 | 1.6 | 1.6 | 1.6 | 1.6 | 1.6 | 1.6 | 1.6 | 1.6 |

Additional peptides were synthesized as described above. Antimicrobial properties of these peptides have been determined and are summarized below.

TABLE 5
Effect of selected antimicrobial sequences on strains of MSSP

| SEQ ID | 71990 | 72036 | 72191 | 76986 | 77378 | 78032 | 81923 | 84250 | 84658 | 86000 | 86001 |
|---|---|---|---|---|---|---|---|---|---|---|---|
| 97 | 3.1 | 3.1 | 3.1 | 1.6 | 1.6 | 3.1 | 3.1 | 3.1 | 3.1 | 1.6 | 1.6 |
| 98 | 1.6 | 1.6 | 1.6 | 1.6 | 1.6 | 1.6 | 1.6 | 1.6 | 1.6 | 1.6 | 1.6 |
| 99 | 1.6 | 1.6 | 3.1 | 1.6 | 1.6 | 1.6 | 1.6 | 3.1 | 3.1 | 3.1 | 1.6 |
| 100 | 1.6 | 1.6 | 3.1 | 1.6 | 3.1 | 1.6 | 1.6 | 1.6 | 1.6 | 1.6 | 1.6 |
| 102 | 1.6 | 1.6 | 3.1 | 1.6 | 0.8 | 1.6 | 1.6 | 1.6 | 1.6 | 1.6 | 1.6 |
| 103 | 3.1 | 3.1 | 3.1 | 3.1 | 3.1 | 3.1 | 3.1 | 6.2 | 3.1 | 3.1 | 3.1 |
| 104 | 1.6 | 3.1 | 1.6 | 1.6 | 1.6 | 1.6 | 3.1 | 3.1 | 1.6 | 3.1 | 1.6 |
| 105 | 1.6 | 1.6 | 1.6 | 1.6 | 0.8 | 1.6 | 1.6 | 1.6 | 3.1 | 1.6 | 1.6 |
| 106 | 1.6 | 1.6 | 1.6 | 1.6 | 0.8 | 1.6 | 1.6 | 1.6 | 1.6 | 1.6 | 1.6 |
| 107 | 1.6 | 1.6 | 1.6 | 1.6 | 1.6 | 1.6 | 1.6 | 1.6 | 1.6 | 1.6 | 1.6 |
| 108 | 3.1 | 3.1 | 3.1 | 3.1 | 3.1 | 3.1 | 3.1 | 3.1 | 3.1 | 3.1 | 3.1 |
| 109 | 1.6 | 1.6 | 1.6 | 1.6 | 1.6 | 1.6 | 1.6 | 1.6 | 1.6 | 1.6 | 1.6 |
| 110 | 1.6 | 3.1 | 3.1 | 1.6 | 1.6 | 1.6 | 3.1 | 3.1 | 3.1 | 3.1 | 3.1 |
| 112 | 1.6 | 1.6 | 1.6 | 1.6 | 0.8 | 1.6 | 1.6 | 1.6 | 1.6 | 1.6 | 1.6 |
| 113 | 1.6 | 1.6 | 1.6 | 1.6 | 1.6 | 1.6 | 1.6 | 1.6 | 1.6 | 1.6 | 1.6 |
| 114 | 1.6 | 3.1 | 3.1 | 1.6 | 1.6 | 3.1 | 3.1 | 3.1 | 3.1 | 3.1 | 1.6 |
| 115 | 1.6 | 1.6 | 1.6 | 1.6 | 0.8 | 1.6 | 1.6 | 1.6 | 1.6 | 1.6 | 1.6 |
| 116 | 1.6 | 1.6 | 1.6 | 1.6 | 1.6 | 1.6 | 1.6 | 1.6 | 1.6 | 1.6 | 1.6 |
| 117 | 1.6 | 1.6 | 1.6 | 0.8 | 0.8 | 1.6 | 1.6 | 1.6 | 1.6 | 1.6 | 1.6 |
| 32 | 1.6 | 1.6 | 1.6 | 1.6 | 1.6 | 1.6 | 1.6 | 3.1 | 3.1 | 1.6 | 1.6 |
| 28 | 1.6 | 1.6 | 1.6 | 0.8 | 1.6 | 1.6 | 1.6 | 1.6 | 1.6 | 1.6 | 1.6 |
| 29 | 1.6 | 1.6 | 1.6 | 1.6 | 0.8 | 0.8 | 0.8 | 0.8 | 1.6 | 1.6 | 1.6 |
| 30 | 0.8 | 0.8 | 1.6 | 0.8 | 0.8 | 0.8 | 0.8 | 0.8 | 1.6 | 1.6 | 1.6 |
| 31 | 1.6 | 1.6 | 1.6 | 1.6 | 1.6 | 1.6 | 1.6 | 1.6 | 1.6 | 1.6 | 1.6 |

TABLE 6
Effect of Antimicrobial peptides on different bacteria.

| | MR | MR | MR | MR | MR | MR | MR | MR | MR | MR | SA | EC | SP |
|---|---|---|---|---|---|---|---|---|---|---|---|---|---|
| SEQ ID | 71994 | 72035 | 72192 | 76923 | 79882 | 80279 | 81926 | 86002 | 87665 | 88493 | 29213 | 25922 | 49051 |
| 97 | 1.6 | 3.1 | 3.1 | 1.6 | 1.6 | 3.1 | 1.6 | 3.1 | 3.1 | 3.1 | 6.2 | 3.1 | 3.1 |
| 98 | 1.6 | 3.1 | 1.6 | 1.6 | 0.8 | 1.6 | 1.6 | 1.6 | 1.6 | 1.6 | 3.1 | 1.6 | 1.6 |
| 99 | 1.6 | 3.1 | 3.1 | 1.6 | 1.6 | 1.6 | 3.1 | 3.1 | 1.6 | 1.6 | 6.2 | 1.6 | 3.1 |
| 100 | 1.6 | 3.1 | 1.6 | 1.6 | 1.6 | 1.6 | 1.6 | 1.6 | 1.6 | 3.1 | 6.2 | 0.8 | 1.6 |
| 102 | 3.1 | 3.1 | 1.6 | 1.6 | 1.6 | 1.6 | 1.6 | 1.6 | 1.6 | 3.1 | 6.2 | 1.6 | 1.6 |
| 103 | 3.1 | 3.1 | 3.1 | 3.1 | 3.1 | 3.1 | 3.1 | 3.1 | 3.1 | 3.1 | 25 | 6.2 | 3.1 |
| 104 | 3.1 | 1.6 | 1.6 | 1.6 | 1.6 | 1.6 | 1.6 | 1.6 | 3.1 | 3.1 | 6.2 | 3.1 | 1.6 |
| 105 | 1.6 | 1.6 | 1.6 | 1.6 | 0.8 | 1.6 | 1.6 | 1.6 | 1.6 | 3.1 | 6.2 | 3.1 | 1.6 |
| 106 | 1.6 | 3.1 | 1.6 | 1.6 | 0.8 | 1.6 | 1.6 | 1.6 | 1.6 | 3.1 | 3.1 | 3.1 | 1.6 |
| 107 | 1.6 | 3.1 | 1.6 | 1.6 | 0.8 | 1.6 | 1.6 | 1.6 | 1.6 | 1.6 | 3.1 | 3.1 | 1.6 |
| 108 | 3.1 | 3.1 | 3.1 | 3.1 | 3.1 | 3.1 | 3.1 | 3.1 | 3.1 | 3.1 | 12.5 | 0.8 | 3.1 |
| 109 | 1.6 | 1.6 | 1.6 | 1.6 | 0.8 | 1.6 | 1.6 | 1.6 | 1.6 | 1.6 | 3.1 | 1.6 | 1.6 |
| 110 | 3.1 | 3.1 | 3.1 | 3.1 | 1.6 | 1.6 | 3.1 | 3.1 | 3.1 | 3.1 | 6.2 | 1.6 | 3.1 |
| 112 | 1.6 | 1.6 | 1.6 | 1.6 | 0.8 | 0.8 | 1.6 | 1.6 | 1.6 | 1.6 | 3.1 | 1.6 | 1.6 |
| 113 | 1.6 | 1.6 | 1.6 | 1.6 | 1.6 | 1.6 | 1.6 | 1.6 | 1.6 | 1.6 | 6.2 | 1.6 | 1.6 |
| 114 | 3.1 | 3.1 | 3.1 | 3.1 | 1.6 | 1.6 | 3.1 | 3.1 | 3.1 | 3.1 | 6.2 | 1.6 | 3.1 |
| 115 | 1.6 | 1.6 | 1.6 | 1.6 | 0.8 | 1.6 | 1.6 | 1.6 | 1.6 | 1.6 | 3.1 | 0.8 | 1.6 |
| 116 | 1.6 | 1.6 | 1.6 | 1.6 | 1.6 | 1.6 | 1.6 | 3.1 | 1.6 | 1.6 | 6.2 | 0.8 | 1.6 |
| 117 | 1.6 | 1.6 | 0.8 | 1.6 | 0.8 | 1.6 | 1.6 | 0.8 | 1.6 | 1.6 | 6.2 | 0.8 | 1.6 |
| 32 | 3.1 | 3.1 | 1.6 | 3.1 | 1.6 | 1.6 | 3.1 | 1.6 | 1.6 | 1.6 | 12.5 | 1.6 | 3.1 |
| 28 | 1.6 | 3.1 | 3.1 | 1.6 | 0.8 | 1.6 | 1.6 | 1.6 | 1.6 | 1.6 | 6.2 | 0.8 | 1.6 |
| 29 | 1.6 | 1.6 | 0.8 | 1.6 | 0.8 | 0.8 | 0.8 | 1.6 | 1.6 | 1.6 | 3.1 | 1.6 | 1.6 |
| 30 | 1.6 | 0.8 | 0.8 | 0.8 | 0.8 | 0.8 | 1.6 | 1.6 | 1.6 | 1.6 | 3.1 | 1.6 | 1.6 |
| 31 | 1.6 | 1.6 | 1.6 | 1.6 | 1.6 | 1.6 | 1.6 | 1.6 | 1.6 | 1.6 | 3.1 | 1.6 | 1.6 |

MR = MRSP,
SA = S. aureus,
EC = E. coli
SP = S. pseudintermedius

Safety of the peptides listed in table 2 was determining by measuring cell viability. Canine-derived epithelial keratinocyte (CPEK) cells were propagated to determine cell viability in the presence of peptides. Cells were grown from a frozen stock in CnT-09-5 (with supplements) pre-warmed media in a T75 flask and incubated overnight at 37° C., 5% CO₂. Cells were washed with phosphate buffer and replenished with pre-warmed CnT-05-9 media and repeated for several days until cells reached a density of 6.6×10⁴ cells/mL. Cells were then transferred to a 384 well plate, allowed to settle and dosed with peptides and melittin control peptide (50 μM to 0.05 μM) and incubated overnight at 37° C., 5% CO₂. 0.1% TritonX100 as (HPE) and phosphate buffer alone as (ZPE) were added to the plates to calculate percent effect once the assay was terminated with 10 μL CELLTITER-GLO® assay reagents for a luminescent readout. The results are provided in Table 7.

TABLE 7

| SEQ ID | CPEK | cRBC |
|---|---|---|
| NO: | (50 μM) | (50 μM) |
| 3 | 42.1 | 67.1 |
| 4 | 16.6 | 67.1 |
| 5 | 28.6 | 3.1 |
| 2 | −1.7 | 64.0 |
| 6 | 4.1 | 1.1 |
| 96 | 51.9 | 101.4 |
| 7 | 11.3 | 84.1 |
| 8 | 24.6 | 30.2 |
| 9 | 52.3 | 101.3 |
| 10 | 31.5 | 101.1 |
| 11 | 22.0 | 57.8 |
| 1 | 21.5 | 20.5 |

Safety of the peptides listed in table 6 was determining by measuring cell viability as described above. The results are provided in Table 8.

TABLE 8

| | cRBC | CPEK |
|---|---|---|
| SEQ ID | (50 μM) | (50 μM) |
| 97 | 31.2 | 12.9 |
| 98 | 35.0 | 9.3 |
| 99 | 100.0 | 19.9 |
| 100 | 100.0 | 29.5 |
| 102 | 23.9 | −2.1 |
| 103 | 7.9 | −0.5 |
| 104 | 100.6 | 6.0 |
| 105 | 97.9 | 8.6 |
| 106 | 101.5 | 25.2 |
| 107 | 101.8 | 26.3 |
| 108 | 27.7 | 9.6 |
| 109 | 99.8 | 19.2 |
| 110 | 46.3 | 10.7 |
| 112 | 61.0 | 14.2 |
| 113 | 86.3 | 15.7 |
| 114 | 97.9 | 11.9 |
| 115 | 94.6 | 5.1 |
| 116 | 87.0 | 16.0 |
| 117 | 97.8 | 14.7 |
| 32 | 98.3 | 9.1 |
| 28 | 94.4 | 19.4 |
| 29 | 68.2 | 17.5 |
| 30 | 63.5 | 17.0 |
| 31 | 43.5 | 11.0 |

These data demonstrate that the antimicrobial peptides of the instant invention are not only effective against the tested strains of bacteria but also safe, particularly for non-systemic, e.g., topical, administration.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1-21. (canceled)

22. The amino acid sequence that is 18-21 amino acids long and comprises, at its N-terminus, a peptide that differs from SEQ ID NO: 1(KWCFRVCYRGICYRRCRD) or SEQ ID NO: 29 (PKWCFRVCYRGICYRRCRD) SEQ ID NO: 1 by one, two, three amino acids, wherein the amino acid at position corresponding to position 18 of SEQ ID NO: 1 is glutamic acid and wherein said peptide optionally further comprises one or two differing amino acids independently selected from the group consisting of
arginine or glycine at position corresponding to position 1 of SEQ ID NO: 1;
phenylalanine or arginine at position corresponding to position 2 of SEQ ID NO: 1;
valine or tryptophan at position corresponding to position 4 of SEQ ID NO: 1;
tyrosine at position corresponding to position 5 of SEQ ID NO: 1;
arginine at position corresponding to position 8 of SEQ ID NO: 1;
glycine at position corresponding to position 9 of SEQ ID NO: 1;
arginine at position corresponding to position 10 of SEQ ID NO: 1;
alanine, phenylalanine, or valine at position corresponding to position 11 of SEQ ID NO: 1;
histidine at position corresponding to position 14 of SEQ ID NO: 1;
lysine at position corresponding to position 15 of SEQ ID NO: 1;
asparagine at position corresponding to position 17 of SEQ ID NO: 1

23. (canceled)

24. The amino acid sequence according to claim 22, comprising asparagine at position corresponding to position 17 of SEQ ID NO: 1.

25. The amino acid sequence according to claim 22, comprising glycine at position corresponding to position 1 of SEQ ID NO: 1.

26. The amino acid sequence according to claim 22, comprising alanine at position corresponding to position 11 of SEQ ID NO: 1.

27. The amino acid sequence according to claim 22, wherein arginine is present at position corresponding to position 14 of SEQ ID NO: 1, at position corresponding to position 15 of SEQ ID NO: 1, or both.

28. The amino acid sequence according to claim 22, comprising arginine at position corresponding to position 14 of SEQ ID NO: 1 and position corresponding to position 15 of SEQ ID NO: 1.

29. The amino acid sequence according to claim 22, wherein said amino acid sequence is 18 amino acids long.

30. The amino acid sequence of claim 22, wherein the peptide differs from SEQ ID NO: 1 by three, two, or one amino acid.

31. (canceled)

32. (canceled).

33. (canceled).

34. A method of treating skin infections in an animal in need thereof, comprising administering to the animal a composition according to claim 53.

35. (canceled).

36. The method of claim 34, wherein said formulation is administered topically.

37. The method of claim 34, wherein the animal is a dog.

38. A method of treating mastitis in an animal in need thereof, comprising administering to the animal a composition according to, claim 53.

39. The method of claim 38 wherein said formulation is administered to the mammary gland of the animal.

40. (canceled)

41. (currently amended A method of treating respiratory infection in an animal in need thereof, comprising administering to the animal a composition according to, claims 53.

42.-49 (canceled)

50. The amino acid sequence of claim 22, wherein the peptide differs from SEQ ID NO: 22 by one amino acid, and wherein said one differing amino acid is glutamic acid at position corresponding to position 18 of SEQ ID NO: 1.

51. The amino acid sequence according to claim 22, wherein said amino acid sequence is 19 amino acids long.

52. The amino acid sequence of claim 51, wherein the peptide differs from SEQ ID NO: 29 by one amino acid, and wherein said one differing amino acid is glutamic acid at position corresponding to position 18 of SEQ ID NO: 1.

53. A composition comprising the amino acid sequence of claim 22 and a pharmaceutically acceptable carrier.


SEQUENCE LISTING (117 sequences)

SEQ ID NO: 1 - protein, 18 aa, synthetic construct
  REGION 1..18: note = Antimicrobial peptide
KWCFRVCYRGICYRRCRD

SEQ ID NO: 2 - protein, 18 aa, synthetic construct
  REGION 1..18: note = Artificial antimicrobial peptide
RWCFRVCYRGICYRRCRD

SEQ ID NO: 3 - protein, 18 aa, synthetic construct
  REGION 1..18: note = Artificial Antimicrobial peptide
GWCFRVCYRGICYRRCRD

SEQ ID NO: 4 - protein, 18 aa, synthetic construct
  REGION 1..18: note = Artificial antimicrobial peptide
KFCFRVCYRGICYRRCRD

SEQ ID NO: 5 - protein, 18 aa, synthetic construct
  REGION 1..18: note = Artificial Antimicrobial peptide
KWCFYVCYRGICYRRCRD

SEQ ID NO: 6 - protein, 18 aa, synthetic construct
  REGION 1..18: note = Artificial Antimicrobial peptide
KWCFRVCRRGICYRRCRD

SEQ ID NO: 7 - protein, 18 aa, synthetic construct
  REGION 1..18: note = Artificial Antimicrobial peptide
KWCFRVCYRGVCYRRCRD

SEQ ID NO: 8 - protein, 18 aa, synthetic construct
  REGION 1..18: note = Artificial Antimicrobial peptide
KWCFRVCYRGACYRRCRD

SEQ ID NO: 9 - protein, 18 aa, synthetic construct
  REGION 1..18: note = Artificial Antimicrobial peptide
KWCFRVCYRGFCYRRCRD

SEQ ID NO: 10 - protein, 18 aa, synthetic construct
  REGION 1..18: note = Artificial Antimicrobial peptide
KWCFRVCYRGICYHRCRD

SEQ ID NO: 11 - protein, 18 aa, synthetic construct
  REGION 1..18: note = Artificial Antimicrobial peptide
KWCFRVCYRGICYRRCND

SEQ ID NO: 12 - protein, 19 aa, synthetic construct
  REGION 1..19: note = Artificial Antimicrobial peptide
  VARIANT 1: note = Absent or Proline
  VARIANT 2: note = Lysine, Arginine, Glycine, or Proline
  VARIANT 3: note = Phenylalanine, Tryptophan or Arginine
  VARIANT 5: note = phenylalanine, valine or tryptophan
  VARIANT 6: note = arginine, tyrosine or phenylalanine
  VARIANT 7: note = valine or alanine
  VARIANT 9: note = tyrosine, arginine, lysine or tryptophan
  VARIANT 10: note = arginine, phenylalanine, or glycine
  VARIANT 11: note = arginine or glycine
  VARIANT 12: note = isoleucine, alanine, phenylalanine, tyrosine or valine
  VARIANT 15: note = arginine or histidine
  VARIANT 16: note = arginine or lysine
  VARIANT 18: note = arginine, lysine, alanine, or asparagine
  VARIANT 19: note = Any amino acid, may be 0-4 amino acids long
XXXCXXXCXXXXCYXXCXX

SEQ ID NO: 13 - protein, 17 aa, synthetic construct
  REGION 1..17: note = Artificial Antimicrobial peptide
KWCFRVCYRGICYRRCR

SEQ ID NO: 14 - skipped

SEQ ID NO: 15 - protein, 4 aa, synthetic construct
  REGION 1..4: note = Artificial Antimicrobial peptide
RRRF

SEQ ID NO: 16 - protein, 17 aa, synthetic construct
  REGION 1..17: note = Artificial Antimicrobial peptide
GFCWYVCYRGICYRRCN

SEQ ID NO: 17 - protein, 4 aa, synthetic construct
  REGION 1..4: note = Artificial Antimicrobial peptide
YRGI

SEQ ID NO: 18 - protein, 4 aa, synthetic construct
  REGION 1..4: note = Artificial Antimicrobial peptide fragment
YRGV

SEQ ID NO: 19 - protein, 4 aa, synthetic construct
  REGION 1..4: note = Artificial Antimicrobial peptide fragment
YRGF

SEQ ID NO: 20-22 - skipped (3 entries)

SEQ ID NO: 23 - protein, 17 aa, synthetic construct
  REGION 1..17: note = Artificial Antimicrobial peptide
KFCVYVCYRGICYRRCK

SEQ ID NO: 24 - protein, 17 aa, synthetic construct
  REGION 1..17: note = Artificial Antimicrobial peptide
KWCFRVCYRGVCYRRCR

SEQ ID NO: 25 - protein, 17 aa, synthetic construct
  REGION 1..17: note = Artificial Antimicrobial peptide
KWCFRVCRRRFCYRRCR

SEQ ID NO: 26 - protein, 17 aa, synthetic construct
  REGION 1..17: note = Artificial Antimicrobial peptide
  SITE 17: note = Mod_res - Amidated
GFCWYVCYRGICYRRCN

SEQ ID NO: 27 - protein, 17 aa, synthetic construct
  REGION 1..17: note = Artificial Antimicrobial peptide
  MOD_RES 17: note = Amidated
GFCWYVCYRGFCYRRCN

SEQ ID NO: 28 - protein, 18 aa, synthetic construct
  REGION 1..18: note = Artificial Antimicrobial peptide
PKWCFRVCYRGICYRRCR

SEQ ID NO: 29 - protein, 19 aa, synthetic construct
  REGION 1..19: note = Artificial Antimicrobial peptide
PKWCFRVCYRGICYRRCRD

SEQ ID NO: 30 - protein, 17 aa, synthetic construct
  REGION 1..17: note = Artificial Antimicrobial peptide
PWCFRVCYRGICYRRCR

SEQ ID NO: 31 - protein, 18 aa, synthetic construct
  REGION 1..18: note = Artificial Antimicrobial peptide
PWCFRVCYRGICYRRCRD

SEQ ID NO: 32 - skipped

SEQ ID NO: 33 - protein, 18 aa, synthetic construct
  REGION 1..18: note = Artificial Antimicrobial peptide
PGFCWYVCRRRFCYRRCN

SEQ ID NO: 34 - protein, 17 aa, synthetic construct
  REGION 1..17: note = Artificial Antimicrobial peptide
PFCWYVCRRRFCYRRCN

SEQ ID NO: 35 - protein, 17 aa, synthetic construct
  REGION 1..17: note = Artificial Antimicrobial peptide
GFCWYVCRRRFCHRRCN

SEQ ID NO: 36 - protein, 17 aa, synthetic construct
  REGION 1..17: note = Artificial Antimicrobial peptide
GVCVYVCRRRFCYRRCN

SEQ ID NO: 37 - protein, 17 aa, synthetic construct
  REGION 1..17: note = Artificial Antimicrobial peptide
GVCVYVCRRRFCYRRCN

SEQ ID NO: 38 - protein, 17 aa, synthetic construct
  REGION 1..17: note = Artificial Antimicrobial peptide
  MOD_RES 17: note = Amidation
GFCWYVCRRRFCYRRCN

SEQ ID NO: 39 - protein, 19 aa, synthetic construct
  REGION 1..19: note = Artificial Antimicrobial peptide
PGFCWYVCRRRFCYRRCND

SEQ ID NO: 40 - protein, 17 aa, synthetic construct
  REGION 1..17: note = Artificial Antimicrobial peptide
GFCWYVCRRRHCYRRCN

SEQ ID NO: 41 - protein, 17 aa, synthetic construct
  REGION 1..17: note = Artificial Antimicrobial peptide
KFCVYVCRRRFCYRRCK

SEQ ID NO: 42 - protein, 17 aa, synthetic construct
  REGION 1..17: note = Artificial Antimicrobial peptide
GHCWYVCRRRFCYRRCN

SEQ ID NO: 43 - protein, 17 aa, synthetic construct
  REGION 1..17: note = Artificial Antimicrobial peptide
GFCWYVCRRRFCYRRCS

SEQ ID NO: 44 - protein, 17 aa, synthetic construct
  REGION 1..17: note = Artificial Antimicrobial peptide
  MOD_RES 1: note = N-Acetylated
  MOD_RES 17: note = Amidated
GFCWYVCYRGICYRRCN

SEQ ID NO: 45 - protein, 17 aa, synthetic construct
  REGION 1..17: note = Artificial Antimicrobial peptide
  SITE 1: note = Mod-res - N-Acetylated
  SITE 17: note = Mod-res - Amidated
GFCWYVCYRGFCYRRCN

SEQ ID NO: 46 - protein, 17 aa, synthetic construct
  REGION 1..17: note = Artificial Antimicrobial peptide
GFCWYVCYRGFCYRRCN

SEQ ID NO: 47 - protein, 17 aa, synthetic construct
  REGION 1..17: note = Artificial Antimicrobial peptide
KFCWRVCRRRFCRRRCN

SEQ ID NO: 48 - protein, 17 aa, synthetic construct
  REGION 1..17: note = Artificial Antimicrobial peptide
GFCWYVCRRGFCYRRCN

SEQ ID NO: 49 - protein, 17 aa, synthetic construct
  REGION 1..17: note = Artificial Antimicrobial peptide
KWCFRVCRNGVCYRRCR

SEQ ID NO: 50 - protein, 17 aa, synthetic construct
  REGION 1..17: note = Artificial Antimicrobial peptide
GFCWYTCRRRFCYRRCN

SEQ ID NO: 51 - protein, 17 aa, synthetic construct
  REGION 1..17: note = Artificial Antimicrobial peptide
GFCWYVCYRGFCHRRCN

SEQ ID NO: 52 - protein, 17 aa, synthetic construct
  REGION 1..17: note = Artificial Antimicrobial peptide
GFCWYVCRRRFCHRRCN

SEQ ID NO: 53 - protein, 17 aa, synthetic construct
  REGION 1..17: note = Artificial Antimicrobial peptide
  SITE 1: note = mod-res - N-Palmitoylated
GFCWYVCRRRFCYRRCN

SEQ ID NO: 54 - protein, 17 aa, synthetic construct
  REGION 1..17: note = Artificial Antimicrobial peptide
KFCWNVCRRRFCHRRCK

SEQ ID NO: 55 - protein, 17 aa, synthetic construct
  REGION 1..17: note = Artificial Antimicrobial peptide
GFCWYVCRRGICYRRCN

SEQ ID NO: 56 - protein, 17 aa, synthetic construct
  REGION 1..17: note = Artificial Antimicrobial peptide
GFCWYVCRRGICYRRCN

SEQ ID NO: 57 - protein, 17 aa, synthetic construct
  REGION 1..17: note = Artificial Antimicrobial peptide
GFCWNVCRRRFCRRRCN

SEQ ID NO: 58 - protein, 17 aa, synthetic construct
  REGION 1..17: note = Artificial Antimicrobial peptide
KFCVNVCYRGICHRRCK

SEQ ID NO: 59 - protein, 17 aa, synthetic construct
  REGION 1..17: note = Artificial Antimicrobial peptide
KFCVNVCRRRFCHRRCK

SEQ ID NO: 60 - protein, 17 aa, synthetic construct
  REGION 1..17: note = Artificial Antimicrobial peptide
KFCVNVCRRRFCRRRCK

SEQ ID NO: 61 - protein, 17 aa, synthetic construct
  REGION 1..17: note = Artificial Antimicrobial peptide
GFCWYVCYRGFCYQQCN

SEQ ID NO: 62 - protein, 17 aa, synthetic construct
  REGION 1..17: note = Artificial Antimicrobial peptide
GFCWYVCYRGFCYDDCN

SEQ ID NO: 63 - protein, 17 aa, synthetic construct
  REGION 1..17: note = Artificial Antimicrobial peptide
GFCWYVCPKGYCYRRCN

SEQ ID NO: 64 - protein, 17 aa, synthetic construct
  REGION 1..17: note = Artificial Antimicrobial peptide
GFCWYVCKNGFCYRRCN

SEQ ID NO: 65 - protein, 14 aa, synthetic construct
  REGION 1..14: note = Artificial Antimicrobial peptide
  SITE 14: note = mod_res - Amidated
RKGCKCKNGFCVCR

SEQ ID NO: 66 - protein, 17 aa, synthetic construct
  REGION 1..17: note = Artificial Antimicrobial peptide
GFCWNVCRRRFCHRRCN

SEQ ID NO: 67 - protein, 17 aa, synthetic construct
  REGION 1..17: note = Artificial Antimicrobial peptide
GFCWNVCRRRFCHRRCN

SEQ ID NO: 68 - protein, 17 aa, synthetic construct
  REGION 1..17: note = Artificial Antimicrobial peptide
GFCWNVCYRGICHRRCN

SEQ ID NO: 69 - protein, 17 aa, synthetic construct
  REGION 1..17: note = Artificial Antimicrobial peptide
KVCVNVCKQGICRKRCK

SEQ ID NO: 70 - protein, 16 aa, synthetic construct
  REGION 1..16: note = Artificial Antimicrobial peptide
GCWYVCRNGVCYRRCN

SEQ ID NO: 71 - protein, 17 aa, synthetic construct
  REGION 1..17: note = Artificial Antimicrobial peptide
GFCWYVCRNGVCYRRCN

SEQ ID NO: 72 - protein, 17 aa, synthetic construct
  REGION 1..17: note = Artificial Antimicrobial peptide
GFCWNVCYRGFCHRRCN

SEQ ID NO: 73 - protein, 17 aa, synthetic construct
  REGION 1..17: note = Artificial Antimicrobial peptide
GFCWYVCYRGFCYHHCN

SEQ ID NO: 74 - protein, 17 aa, synthetic construct
  REGION 1..17: note = Artificial Antimicrobial peptide
NVCVVRCRRGFCNRRCK

SEQ ID NO: 75 - protein, 16 aa, synthetic construct
  REGION 1..16: note = Artificial Antimicrobial peptide
KCVRVCRRRACRRRCK

SEQ ID NO: 76 - protein, 16 aa, synthetic construct
  REGION 1..16: note = Artificial Antimicrobial peptide
KCVRVCRRGFCNRRCK

SEQ ID NO: 77 - protein, 17 aa, synthetic construct
  REGION 1..17: note = Artificial Antimicrobial peptide
GFCWYVCKNGYCYRRCN

SEQ ID NO: 78 - protein, 17 aa, synthetic construct
  REGION 1..17: note = Artificial Antimicrobial peptide
GFCWYVCRNGYCYRRCN

SEQ ID NO: 79 - protein, 17 aa, synthetic construct
  REGION 1..17: note = Artificial Antimicrobial peptide
GFCWYVCRNGYCHRRCN

SEQ ID NO: 80 - protein, 17 aa, synthetic construct
  REGION 1..17: note = Artificial Antimicrobial peptide
GFCWYVCYRGICHRRCN

SEQ ID NO: 81 - protein, 16 aa, synthetic construct
  REGION 1..16: note = Artificial Antimicrobial peptide
KCVRVCRRGFCNRRCK

SEQ ID NO: 82 - protein, 17 aa, synthetic construct
  REGION 1..17: note = Artificial Antimicrobial peptide
GFCRYVCYRGICRRRCN

SEQ ID NO: 83 - protein, 17 aa, synthetic construct
  REGION 1..17: note = Artificial Antimicrobial peptide
KFCVNVCRNGICRRRCK

SEQ ID NO: 84 - protein, 17 aa, synthetic construct
  REGION 1..17: note = Artificial Antimicrobial peptide
GVCVNVCRRRFCHRRCN

SEQ ID NO: 85 - protein, 18 aa, synthetic construct
  REGION 1..18: note = Artificial Antimicrobial peptide
KKVCVNVCKQGICHKRCK

SEQ ID NO: 86 - protein, 18 aa, synthetic construct
  REGION 1..18: note = Artificial Antimicrobial peptide
KKVCVNVCRQGICHRRCK

SEQ ID NO: 87 - protein, 17 aa, synthetic construct
  REGION 1..17: note = Artificial Antimicrobial peptide
GFCRYVCRRGICRRRCN

SEQ ID NO: 88 - protein, 17 aa, synthetic construct
  REGION 1..17: note = Artificial Antimicrobial peptide
KFCVNVCYRGICRRRCK

SEQ ID NO: 89 - protein, 16 aa, synthetic construct
  REGION 1..16: note = Artificial Antimicrobial peptide
KCRRVCRRGFCYVVCN

SEQ ID NO: 90 - protein, 17 aa, synthetic construct
  REGION 1..17: note = Artificial Antimicrobial peptide
GHCHHVCRRRHCHRRCN

SEQ ID NO: 91 - protein, 17 aa, synthetic construct
  REGION 1..17: note = Artificial Antimicrobial peptide
GFCWYVCYRGFCYRRCN

SEQ ID NO: 92 - protein, 18 aa, synthetic construct
  REGION 1..18: note = Artificial Antimicrobial peptide
RGGRLCYCRRRFCVCVGR

SEQ ID NO: 93 - protein, 18 aa, synthetic construct
  REGION 1..18: note = Artificial Antimicrobial peptide
RRWCFRVCYRGFCYRKCR

SEQ ID NO: 94 - protein, 17 aa, synthetic construct
  REGION 1..17: note = Artificial Antimicrobial peptide
GFCWYVCRRRFCYRRCN

SEQ ID NO: 95 - protein, 17 aa, synthetic construct
  REGION 1..17: note = Artificial Antimicrobial peptide
GFCWYVCRRRFCYRRCN

SEQ ID NO: 96 - protein, 18 aa, synthetic construct
  REGION 1..18: note = Artificial Antimicrobial peptide
KWCFRVCYGRICYRRCRD

SEQ ID NO: 97 - protein, 18 aa, synthetic construct
  REGION 1..18: note = Artificial Antimicrobial peptide
KRCFRVCYRGICYRRCRD

SEQ ID NO: 98 - protein, 18 aa, synthetic construct
  REGION 1..18: note = Artificial Antimicrobial peptide
KWCVRVCYRGICYRRCRD

SEQ ID NO: 99 - protein, 18 aa, synthetic construct
  REGION 1..18: note = Artificial Antimicrobial peptide
KWCFFVCYRGICYRRCRD

SEQ ID NO: 100 - protein, 18 aa, synthetic construct
  REGION 1..18: note = Artificial Antimicrobial peptide
KWCFWVCYRGICYRRCRD

SEQ ID NO: 101 - protein, 19 aa, synthetic construct
  REGION 1..19: note = Artificial Antimicrobial peptide
KWCFRVYCYRGICYRRCRD

SEQ ID NO: 102 - protein, 18 aa, synthetic construct
  REGION 1..18: note = Artificial Antimicrobial peptide
KWCFRACYRGICYRRCRD

SEQ ID NO: 103 - protein, 18 aa, synthetic construct
  REGION 1..18: note = Artificial Antimicrobial peptide
KWCFRVCKRGICYRRCRD

SEQ ID NO: 104 - protein, 18 aa, synthetic construct
  REGION 1..18: note = Artificial Antimicrobial peptide
KWCFRVCYFGICYRRCRD

SEQ ID NO: 105 - protein, 18 aa, synthetic construct
  REGION 1..18: note = Artificial Antimicrobial peptide
KWCFRVCYRGICYRRCRN

SEQ ID NO: 106 - protein, 18 aa, synthetic construct
  REGION 1..18: note = Artificial Antimicrobial peptide
KWCWRVCYRGICYRRCRD

SEQ ID NO: 107 - protein, 18 aa, synthetic construct
  REGION 1..18: note = Artificial Antimicrobial peptide
KWCFRVCWRGICYRRCRD

SEQ ID NO: 108 - protein, 18 aa, synthetic construct
  REGION 1..18: note = Artificial Antimicrobial peptide
KWCFRVCYGGICYRRCRD

SEQ ID NO: 109 - protein, 18 aa, synthetic construct
  REGION 1..18: note = Artificial Antimicrobial peptide
KWCFRVCYRRICYRRCRD

SEQ ID NO: 110 - protein, 18 aa, synthetic construct
  REGION 1..18: note = Artificial Antimicrobial peptide
KWCFRVCYRGYCYRRCRD

SEQ ID NO: 111 - protein, 19 aa, synthetic construct
  REGION 1..19: note = Artificial Antimicrobial peptide
KWCFRVCYRGICRYRRCRD

SEQ ID NO: 112 - protein, 18 aa, synthetic construct
  REGION 1..18: note = Artificial Antimicrobial peptide
KWCFRVCYRGICYRRCKD

SEQ ID NO: 113 - protein, 18 aa, synthetic construct
  REGION 1..18: note = Artificial Antimicrobial peptide
KWCFRVCYRGICYRRCAD

SEQ ID NO: 114 - protein, 18 aa, synthetic construct
  REGION 1..18: note = Artificial Antimicrobial peptide
KWCFRVCYRGICYRRCRR

SEQ ID NO: 115 - protein, 18 aa, synthetic construct
  REGION 1..18: note = Artificial Antimicrobial peptide
GWCFRVCYRGICYRRCND

SEQ ID NO: 116 - protein, 18 aa, synthetic construct
  REGION 1..18: note = Artificial Antimicrobial peptide
KWCFYVCYRGICYRRCND

SEQ ID NO: 117 - protein, 18 aa, synthetic construct
  REGION 1..18: note = Artificial Antimicrobial peptide
GWCFYVCYRGICYRRCRD